(19) United States
(12) Patent Application Publication
(10) Pub. No.: US 2016/0319318 A1
(43) Pub. Date: Nov. 3, 2016

(54) PROCESS FOR THE PREPARATION OF TIACUMICIN

(71) Applicant: XELLIA PHARMACEUTICALS APS, København S (DK)

(72) Inventors: Espen Fridtjof HANSEN, Lommedalen (NO); Havard KLEMSDAL, Oslo (NO)

(21) Appl. No.: 15/104,604

(22) PCT Filed: Dec. 18, 2014
(86) PCT No.: PCT/EP2014/078552

§ 371 Date: Jun. 15, 2016

(30) Foreign Application Priority Data
Dec. 20, 2013 (NO) 20131718

(51) Int. Cl. C12P 19/62

(52) U.S. Cl. CPC C12P 19/62

(57) ABSTRACT

The present invention relates to the preparation of Tiacumicin B from a Dactylosporangium or Actinoplanes strain capable of producing Tiacumicin B utilizing spray drying and further extraction of the spray dried powder.

The present invention relates to an improved process for the preparation and purification of Tiacumicin B. Specifically, the invention relates to a purification of Tiacumicin B from a dried fermentation broth, followed by extraction and chromatography techniques. The process according to the invention is simpler than the processes according to the prior art, and can easily be used on a large scale for commercial production.

Tiacumicin B can be produced as disclosed in U.S. Pat. No. 4,918,174 or WO2004014295.

### SUMMARY OF THE INVENTION

The present invention concerns a process for preparing Tiacumicin B comprising the steps of drying a fermentation broth prior to extraction with a suitable solvent.

In particular, the present invention provides a process for the preparation of Tiacumicin B comprising the steps of:
a) fermentation of a Tiacumicin B producing strain;
b) drying the fermentation broth;
c) extraction of the dried material with a solvent.

The drying step b) according to the present invention may be performed by spray drying or freeze drying.

According to one embodiment, the solvent used according to the present method is selected from the group consisting of C₁-C₅ alcohols, C₄-C₆ ethers, C₂-C₅ ketones or C₂-C₅ esters.

According to yet an embodiment, the solvent is selected from the group consisting of methanol, ethanol, isopropanol and acetone.

The solvent used according to the present method may furthermore comprise water, such as e.g. at least 1-40% v/v water, such as e.g. at least 10-30% v/v water, or such as e.g. at least 15-20% v/v water. Such solvents, are referred to in the present disclosure as aqueous solvents.

According to one embodiment, a process for preparing Tiacumicin B is provided, comprising the steps of:
a) fermentation of a Tiacumicin B producing strain;
b) drying the fermentation broth;
c) extraction of the dried material with an aqueous solvent comprising 50-90% v/v of an organic solvent selected from the group consisting of methanol, ethanol. isopropanol and acetone.

According to another embodiment, a process is provided comprising the steps of:
a) fermentation of a Dactylosporangium or Actinoplanes strain capable of producing Tiacumicin B;
b) drying the fermentation broth;
c) extraction of the dried material with an aqueous solvent comprising 60-90% v/v methanol and ethanol.

The extraction may be performed with a solvent selected from the group consisting of methanol, ethanol, isopropanol and acetone. According to one embodiment, the solvent is an aqueous solvent comprising 50-90% v/v methanol, ethanol, isopropanol or acetone.

The solvent used according to the present invention may according to one embodiment be an aqueous solvent comprising 60-90% v/v, preferably 70-80% v/v, more preferably 75% v/v of an organic solvent. Said solvent may be selected from the group consisting of methanol, ethanol, isopropanol and acetone.

According to yet another embodiment, the drying is performed by spray drying and the aqueous solvent used in the extraction step is 70-80% v/v methanol. According to yet another embodiment, the drying is performed by spray drying and the aqueous solvent used in the extraction step is 70-80% v/v ethanol.

According to yet another embodiment, the drying is performed by freeze drying and the aqueous solvent used in the extraction step is 70-80% v/v methanol. According to yet another embodiment, the drying is performed by freeze drying and the aqueous solvent used in the extraction step is 70-80% v/v ethanol.

The ratio between solvent volume to dried fermentation broth mass according to the present method may be 1-6 ml/g. According to one embodiment of the present invention, the ratio between solvent volume to dried fermentation broth mass is 2-4 ml/g.

According to the present invention, the Tiacumicin B producing strain to be used in the fermentation step may be selected from the group consisting of Dactylosporangium and Actinoplanes.

The various aspects and more of the present invention, including various embodiments, will be described in further detail, with reference to the detailed description, examples and appended drawings.

### FIGURES

FIG. 1 shows the extraction efficiency at different solvent concentration of a spray dried fermentate.

FIG. 2 shows the extraction yield at different methanol concentration of a spray dried fermentate.

FIG. 3 shows the yield when extracting a spray dried fermentate at different volumes of 75% v/v aqueous methanol.

### DETAILED DESCRIPTION

According to the present invention, any Tiacumicin B producing bacterial strain may be used to provide the fermentation broth according to the present invention. According to one embodiment, Tiacumicin B can be produced by fermentation of Dactylosporangium aurantiacum subspecies hamdenensis NRRL 18085 or Actinoplanes deccanensis ATCC 21983.

The fermentation broth is the liquid nutrient medium comprising the bacterial biomass and the Tiacumicin compounds.

The drying of the fermentation broth can be performed by freeze drying. A preferred freeze drying process includes reducing the temperature to −40° C. and the pressure to 200 mTorr, then performing a primary drying at a temperature in the range −5° C. to 5° C. and thereafter secondary drying at a temperature of about 20° C.

The preferred drying process for the fermentation broth according to the present invention is spray drying. Despite the high temperature in the spray drying process, Tiacumicin B can be recovered in high yield.

A preferred spray drying process includes an in-let temperature of 180-220° C. and an out-let temperature of 80-100° C. The drying is performed to achieve a water content of 0%-12% w/w, more preferred is a water content of less than 10% w/w; e.g. 2%-8% w/w.

C₁-C₅ alcohols are meant to embrace the aliphatic alcohols having 1, 2, 3, 4 or 5 carbon atoms.

C₄-C₆ ethers are meant to embrace the aliphatic ethers having 4, 5 or 6 carbon atoms.

C₂-C₅ ketones are meant to embrace the aliphatic ketones having 2, 3, 4 or 5 carbon atoms.

C₂-C₅ esters are meant to embrace the aliphatic esters having 1, 2, 3, 4 or 5 carbon atoms.

According to one embodiment, the dried fermentation broth is extracted with a solvent selected from the group consisting of methanol, ethanol, isopropanol and acetone.

According to another embodiment, the dried fermentation broth is extracted with an aqueous solvent comprising 50-90% v/v of an organic solvent selected from the group consisting of methanol, ethanol, isopropanol and acetone, preferably 70-80% v/v, more preferably 75% v/v of an organic solvent selected from the group consisting of methanol, ethanol, isopropanol and acetone. The preferred pH during extraction is about 5-7, preferably 6-7, more preferably 7. The preferred temperature during extraction is 20-25° C.

The methanol or ethanol concentration in the extraction solution is subsequently adjusted by evaporation or dilution to a concentration suitable for binding of tiacumicin B on an adsorption resin, e.g. 50% organic solvent in water. After binding on an adsorption resin, the resin is washed with an aqueous solvent solution to remove unbound impurities, e.g. 50% organic solvent. The bound tiacumicin B can be eluted with a solvent comprising 60-100% organic solvent and 0-40 v/v water.

### Experimental Data:

### EXAMPLE 1

Dactylosporangium aurantiacum subspecies hamdenensis NRRL 18085 fermentate was spray dried directly using a Niro Mobile Minor spray dryer with a 2-fluid nozzle. The air pressure was 1.2 bar. Inlet-temperature was ca. 200° C. and outlet temperature was 90-92° C. The feeding rate was 0.5-1.0 liters per hour.

The spray dried powder was divided into eight 5m1 volumetric flasks with ca. 1 gin each. Solvent was then added up to the 5 ml mark. Different solvents were used: ethanol, methanol, isopropanol, n-propanol, acetonitrile, acetone, dimethyl sulfoxide, water. The flasks were shaken in an overhead stirrer for one hour. The slurry was withdrawn and centrifuged. The resulting supernatant was analyzed by HPLC. DMSO was the most efficient solvent in the extraction. However, DMSO is not suitable for production scale due to e.g. high boiling point. Surprisingly, methanol gave highest yield of the other solvents. The yield was 50-100% higher compared to ethanol, isopropanol, n-propanol, acetonitrile and acetone.

### EXAMPLE 2

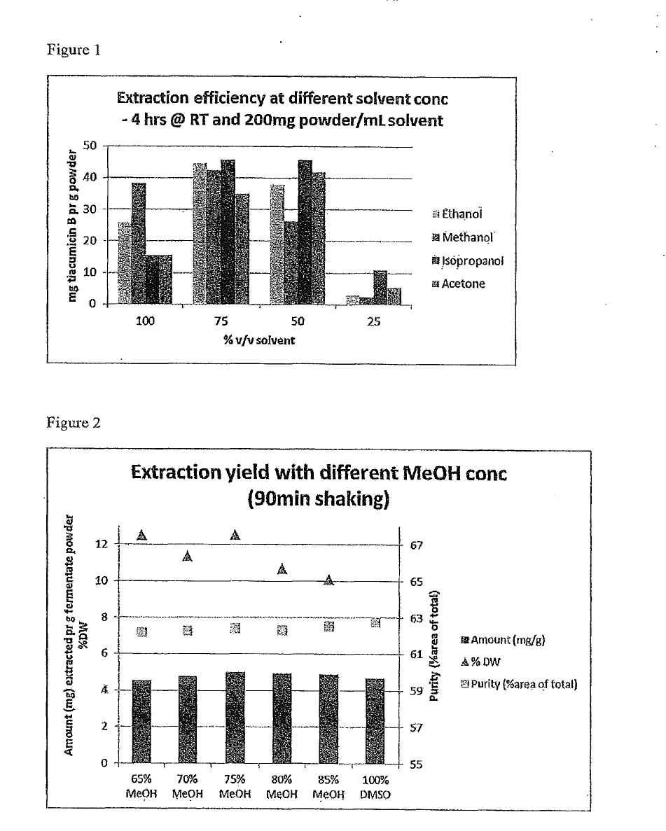

Spray dried Dactylosporangium aurantiacum subspecies hamdenensis NRRL 18085 fermentate was extracted with different solvents and different solvent/water compositions. Ethanol, methanol, isopropanol and acetone were used with concentrations of 100%, 75%, 50% and 25% in water. Five times the weight of the dry fermentate was used in volume (i.e. 200 mg powder pr. ml solution). The slurries were shaken in an overhead shaker for four hours. The slurry was centrifuged, and the supernatant was analyzed by HPLC. The results are shown in FIG. 1.

### EXAMPLE 3

Spray dried Dactylosporangium aurantiacum subspecies hamdenensis NRRL 18085 fermentate was extracted with methanol of different concentrations in water. 1 g fermentate was transferred to a 5 ml measuring flask, and methanol/water solution was added to the mark. Concentrations of 65%, 70%, 75%, 80% and 85% methanol in purified water (RO) were tested. The slurries were shaken in an overhead shaker for 1.5 hours. The slurries were centrifuged, and the supernatants were analyzed by HPLC. The dry weights of the supernatants were also determined.

The results are shown in FIG. 2.

There was a peak in the extraction yield at 75-80% methanol. The dry weight decreased with increasing methanol concentration. The HPLC purity remained the same with all methanol concentrations.

### EXAMPLE 4

Spray dried Dactylosporangium aurantiacum subspecies hamdenensis NRRL 18085 fermentatewas extracted with different volumes of 75% methanol/water. 1 g of fermentate was added 2, 3, 4, 5, and 10 ml 75% methanol in different tubes. The slurries were shaken in an overhead shaker for 1.5 hours. The slurries were centrifuged, and the supernatants were analyzed by HPLC.

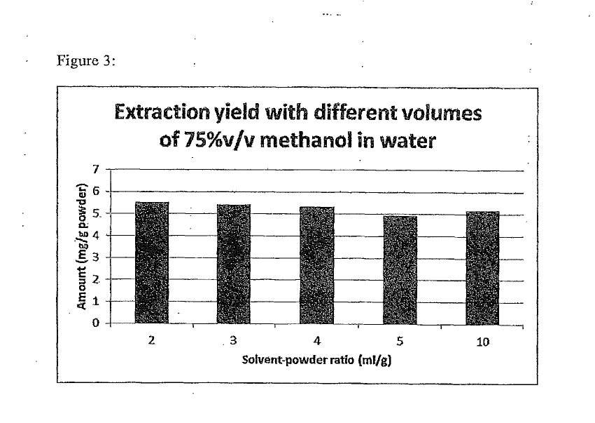

The results are shown in FIG. 3. The yield was similar for all volumes.

### EXAMPLE 5

Spray dried Dactylosporangium aurantiacum subspecies hamdenensis NRRL 18085 fermentatewas extracted with 75% methanol/water (3 times the weight in volume) under stirring for two hours. The slurry was centrifuged, and concentrated two times in a rotary evaporator to give 50% methanol in the solution. The concentrate was loaded directly on a HP20 resin packed in a column. The column was washed with 50% methanol and eluted with a gradient from 50% to 100% methanol. The yield was 92%.

### EXAMPLE 6

The drying step of the present invention may be performed by freeze drying both in small and large scale as shown in the below examples.

### 6.1 Small Scale Freeze Drying

4 L Dactylosporangium aurantiacum subspecies hamdenensis NRRL 18085 fermentate was poured into to steel trays (height of liquid 1.5-2.0 cm). The trays were mounted in a Virtis Genesis 12ES freeze dryer and frozen to −40° C. After reaching the mentioned temperature they were let standing for two hours. Then the condenser was cooled to ea −50° C. and a vacuum of 200 mTorr was established. The shelf temperature was adjusted to −5° C. during 600 min. Then the shelf temperature was quickly adjusted to 0° C. and kept for 1250 min. Another quick adjustment of the shelf temperature to +5° C. was performed, and the product was kept at this temperature for 600 min. The secondary drying was performed by quick adjustment to +20° C. upon which the product was left for 1250 min at vacuum (200 mTorr). Then the tanks were removed from the freeze dryer and 179 g dry material was obtained.

### 6.2 Large Scale Freeze Drying

Ca 110 L Dactylosporangium aurantiacum subspecies hamdenensis NRRL 18085 fermentate was poured into 48 steel trays (height of liquid 1.5-2.0 cm).The trays were placed in two freeze dryers, and frozen to −20° C. After reaching the mentioned temperature, the fermentate was left standing for a while. The condenser was cooled to ca −40° C. Then a vacuum of ca 200 mTorr was established. The shelf temperature was adjusted to +40° C. during ca 44 hours. Then the trays were removed from the freeze dryer and 6.6 kg dry material was obtained.

400 g of freeze dried Dactylosporangium aurantiacum subspecies hamdenensis NRRL 18085 fermentate was added 2000 ml 80% v/v methanol-water and stirred for 1 hour. Then the mixture was centrifuged at 4500 rpm for 15 min. The supernatant was decanted (volume of 1550 ml). The sediment was added 1400 ml 80% v/v methanol-water. The mixture was left over night and stirred for 1 hour the next day. Then it was centrifuged at 4500 rpm for 15min The supernatant was decanted (volume of 1380 ml). Both supernatants were combined to a total volume of 2930 ml. Total yield 79%.

The solution was then evaporated under reduced pressure on a 40° C. water bath. The final volume was 1220 ml comprising 40% water as measured by Karl Fisher titration. This solution was loaded on a column packed with a HP20 resin.

1. A process for preparing Tiacumicin B comprising the steps of:
a) fermentation of a Tiacumicin B producing strain;
b) drying the fermentation broth;
c) extraction of the dried material with an aqueous solvent comprising 50-90% v/v of an organic solvent selected from the group consisting of methanol, ethanol, isopropanol and acetone.

2. A process according to claim 1, wherein the bacterial strain belongs to a species selected from the group consisting of Dactylosporangium and Actinoplanes.

3. A process according to any of the claims 1-2, wherein the aqueous solvent comprises 60-90% v/v, preferably 70-80% v/v, more preferably 75% v/v of an organic solvent selected from the group consisting of methanol, ethanol, isopropanol and acetone.

4. A process according to any of the claims 1-2, comprising the steps of:
a) fermentation of a Dactylosporangium or Actinoplanes strain capable of producing Tiacumicin B;
b) drying the fermentation broth;
c) extraction of the dried material with an aqueous solvent comprising 60-90% v/v methanol and ethanol.

5. A process according to any of the above claims, wherein the drying is freeze drying.

6. A process according to any of the above claims, wherein the drying is spray drying.

7. A process according to claim 4, wherein the drying is spray drying and the aqueous solvent is 70-80% v/v methanol.

8. A process according to claim 4, wherein the drying is spray drying and the aqueous solvent is 70-80% v/v ethanol.

9. A process according to any of claim 1-8, wherein the ratio between solvent volume to dried fermentation broth mass is 1-6 ml/g.

10. A process according to claim 9, wherein the ratio between solvent volume to dried fermentation broth mass is 2-4 ml/g.

11. A process according to any of the above claims, further comprising a chromatography step involving loading of the extraction solution on a column comprising a hydrophobic adsorbent resin, optionally washing before elution.

12. A process according to claim 8 wherein the hydrophobic adsorbent resin is HP20, HP21, HP20SS, SP20, SP20SS, SP825, SP850, SP207, XAD2, XAD7HP, XAD16, XAD16HP, XAD1600, XAD 1180, IRC50 or XAD18.

13. A process for the preparation of Tiacumicin B comprising the steps of:
d) fermentation of Tiacumicin B producing strain;
e) spray drying the fermentation broth;
f) extraction of the dried material with a solvent.

14. A process according to claim 13, wherein the solvent is selected from the group consisting of methanol, ethanol, isopropanol and acetone.

15. A process according to claim 13, wherein the solvent is a 50-90% v/v aqueous solvent selected from the group consisting of methanol, ethanol, isopropanol and acetone.

16. A process according to claim 12, wherein the solvent is an aqueous solvent comprising 50-90% v/v of an organic solvent selected from the group consisting of methanol and ethanol, preferably 70-80% v/v, more preferably 75% v/v of said organic solvent.

17. A process according to any of the claims 14-16, wherein the bacterial strain belongs to a species selected from the group consisting of Dactylosporangium and Actinoplanes.
